(19) United States
(12) Patent Application Publication
(10) Pub. No.: US 2018/0116628 A1
(43) Pub. Date: May 3, 2018

(54) METHOD AND APPARATUS FOR DETECTING INSTANTANEOUS FETAL HEART RATE OF DOPPLER FETAL HEART SOUND BASED ON TIME-FREQUENCY ANALYSIS

(71) Applicant: Guangdong University of Technology, Guangzhou City (CN)

(72) Inventors: Kan Xie, Guangzhou City (CN); Haochuan Zhang, Guangzhou City (CN); Shengli Xie, Guangzhou City (CN); Kun Cai, Guangzhou City (CN)

(21) Appl. No.: 15/335,564

(22) Filed: Oct. 27, 2016

(51) Int. Cl. A61B 8/02; A61B 8/08

(52) U.S. Cl. CPC A61B 8/02; A61B 8/488; A61B 8/0866; A61B 8/5223; A61B 8/5207

(57) ABSTRACT

The present disclosure relates to medical monitoring and provides a method and an apparatus for detecting an instantaneous fetal heart rate of a Doppler fetal heart sound based on time-frequency analysis. The method comprises: pre-processing a Doppler fetal heart sound using a band pass filter; applying time-frequency analysis to the pre-processed ultrasound Doppler fetal heart sound, so as to obtain a time-frequency graph of the ultrasound Doppler fetal heart sound by STFT for simple and fast calculation; applying a cross correlation method to obtain an instantaneous of the fetal heart sound by: selecting a characteristic band from the time-frequency graph of the Doppler fetal heart sound, selecting a characteristic template based on a priori knowledge of the heart sound signal, calculating a cross-correlation function between the characteristic band and the characteristic template to plotting a cross correlation curve; and calculating an instantaneous heart rate of the ultrasound Doppler fetal heart sound signal by calculating intervals between peaks of the cross correlation curve. According to the present disclosure, the instantaneous heart rate of the ultrasound Doppler fetal heart sound signal as collected clinically can be calculated with a simple method and has a fast operation speed and a high accuracy.

### TECHNICAL FIELD

The present disclosure relates to medical monitoring, and more particularly, to a method and an apparatus for detecting an instantaneous fetal heart rate of a Doppler fetal heart sound based on time-frequency analysis.

### BACKGROUND

Fetal heart monitoring is a common method for fetal monitoring that evaluates a fetus' condition in a uterus by monitoring its fetal heart rate. By monitoring fetuses during the perinatal period, it is possible to greatly reduce distresses due to hypoxia or ischemia and reduce birth defects or even deaths of the fetuses, while learning the growth condition of the fetus. Birth defects have now become a severe problem that influences the population quality of this country. Hence, it is of great significance to improve birth qualities by closely monitoring variations in fetal heart rates. As early as the beginning of the 19^{th} century, obstetricians evaluated conditions of fetuses in uteruses by auscultation of hearts. With the development of ultrasound Doppler techniques, Electronic Fetal Monitoring (EFM) during parturition has now become the most popular method for fetal monitoring. The ultrasound Doppler measurement method is currently the most popular method for measuring fetal heart rate.

However, an ultrasound Doppler sound detected by an ultrasound transducer contains widely distributed noise interferences having high amplitudes. When the fetus' body is moving within the mother's body, the strength of the sound signal varies dramatically. In time domain and frequency domain, these interferences are mixed together, which has a great impact on calculation of the instantaneous heart rate of the fetal heart sound signal. Thus, it is important both theoretically and clinically to study how to measure the instantaneous heart rate of the fetal heart sound within the mother's body accurately and efficiently.

Researches on fetal heart monitoring and instantaneous fetal heart rate have been lasted for a long time and there are various processing methods which can be mainly divided into several categories as follows:

(1) Calculation of fetal heart rate based on matched filtering: The basic concept of this method is to use the electrocardios of the mother as obtained previously as a template to cancel electrocardio components of the mother from an abdomen sample signal and extract the electrocardio of the fetus. Since the subtraction of the template from the abdomen signal requires a high accuracy, various measures need to be taken in template calculation and phase and amplitude modifications to ensure the accuracy of the electrocardio of the mother. This is a method based on electrocardio patterns.

(2) Calculation of fetal heart rate based on auto-correlation: It is well known that the correlation method is to extract a known waveform from an additive noise and works well especially for deterministic periodical signals. The effect of the auto-correlation method in extraction of a fetal heart rate signal is not good enough, since the fetal heart rate signal is a repetitive signal, but not a deterministic periodical signal. Further, the fetal heart sound signal does not have an invariant waveform, but has randomly varying period and waveform. Hence, it is difficult to detect the waveform of the auto-correlation function. This is a method based on heart sound pattern.

A normal heart has four heart sounds: a first heart sound (S1), a second heart sound (S2), a third heart sound (S3) and a fourth heart sound (S4). However, in most of cases, only the first and second heart sounds can be heard. The presence of the first heart sound indicates a start of a systolic period and the presence of the second heart sound indicates a start of a diastolic period. The systolic period is defined as a period from the presence of the first heart sound to the presence of the second heart sound. The diastolic period is defined as a period from the presence of the second heart sound to the presence of the first heart sound in the next cardiac cycle. In a cardiac cycle, the major components of the heart sound include a first heart sound, a systolic period, a second heart sound, and a diastolic period, which can fully describe temporal characteristics of the heart sound. For a normal human, typically the systolic period is shorter than the diastolic period. A fetus has on average a heart rate of 120-160 beats per minute and a cardiac cycle of approximately 0.5 seconds, in which the systolic period is about 0.2 seconds and the diastolic period is about 0.3 seconds. That is, in a heart sound signal of a normal human, there is an interval of about 0.2 seconds between the S1 sound and the S2 sound in time domain.

Since the fetal heart sound signal is not a stationary signal, the conventional Fourier transform method cannot describe its frequency components at any time instant and thus cannot analyze it comprehensively. Time-frequency analysis is a powerful tool for analyzing non-stationary signals. This method can convert a one-dimensional signal to a two-dimensional time-frequency plane and provide joint distribution information of the time domain and the frequency domain, which clearly describes a relation between frequency and time of the signal.

### SUMMARY

It is a major object of the present disclosure to overcome the drawbacks of the conventional solutions for detection of fetal heart rate by providing a method for detecting an instantaneous fetal heart rate of a Doppler fetal heart sound based on time-frequency analysis. This detection method jointly uses distribution information of the fetal heart sound in time domain and frequency domain, along with a priori information of the heart sound signal (i.e., the interval between the S1 sound and the S2 sound in the heart sound signal in the time domain of an observation signal), to detect the instantaneous heart rate of the fetal heart sound.

In order to solve the above technical problems, the following solutions are provided.

A method for detecting an instantaneous fetal heart rate of a Doppler fetal heart sound based on time-frequency analysis is provided. The method comprises steps of:

S1—signal pre-processing: applying a band pass filter to a collected Doppler fetal heart sound, the band pass filter having a pass band from f_{L} to f_{H};

S2—time-frequency analysis: applying time-frequency analysis to the Doppler fetal heart sound pre-processed in the step S1 to obtain a time-frequency graph;

S3—characteristic band and template selection: selecting a characteristic band, from f_{CL} to f_{CH}, in the signal from the time-frequency graph, and selecting a time-frequency block containing features of S1 sound and S2 sound from the time-frequency graph, the time-frequency block having a time interval of 0.2 seconds<t₀<0.5 seconds;

S4—cross-correlation function calculation: calculating a cross-correlation function between the characteristic band and a template and plotting a correlation curve based on a result of the cross-correlation function;

S5: calculating a peak of the cross correlation curve by means of threshold detection; and

S6: calculating an instantaneous heart rate value by calculating a differential of the peak, and plotting an instantaneous heart rate graph based on the instantaneous heart rate value.

Further, in the step S1, f_{L} is 50 Hz and f_{H} is 250 Hz, and the band pass filter has the pass band of 50-250 Hz.

Further, in the step S2, the time-frequency analysis is performed by utilizing a Short Time Fourier Transform (STFT) defined as:

s(w,t)=1/2π∫_{-∞}^{+∞}e^{-iw}x(τ)h(τ−t)dτ (1)

where h(t) is a window function, x(τ) is a signal and r is a signal argument, t is a time variable and w is a frequency argument, wherein, by moving an analysis window along a time axis, the resulting two-dimensional time-frequency graph is represented as s(w,t).

Further, in the step S3, the characteristic band is 200-400 Hz, and f_{CL} is 200 Hz and f_{CH} is 400 Hz.

Further, in the step S4, the correlation curve is plotted by utilizing a two-dimensional cross correlation function as:

$$C ( i , j ) = {\sum }_{m = 0}^{Ma - 1} {\sum }_{n = 0}^{Na - 1} A ( m , n ) \cdot conj ( B ( m + i , n + j ) ) \qquad ( 2 )$$

where A is a Ma×Na matrix, B is a Mb×Nb matrix, conj(B) denotes a conjugate of B, 0≤i<Ma+Mb−1, 0≤j<Na+Nb−1, and C(i,j) denotes the cross correlation curve.

Further, in the step S5, the peak of the cross correlation curve is calculated by means of threshold detection, wherein the threshold is:

threshold=param×max{R(n)},

wherein param is 0.9 or a value close to 0.9, and R(n) denotes the cross correlation curve.

Further, in the step S6, the instantaneous heart rate is calculated as:

$$\begin{matrix}Instantaneous Heart Rate = \frac{60}{Time Interval between Two Adjacent Peaks ( seconds )} ( beats \text{/} second ) & ( 3 ) \\ or & \\ Instantaneous Heart Rate = \frac{6000}{Time Interval between Two Adjacent Peaks ( ms )} ( beats \text{/} min ) & ( 4 )\end{matrix}$$

Another object of the present disclosure is to provide an apparatus for applying the method for detecting an instantaneous fetal heart rate of a Doppler fetal heart sound based on time-frequency analysis, capable of obtain the instantaneous heart rate from the fetal heart sound signal accurately. The apparatus comprises: a signal pre-processing module configured to apply a band pass filter to a collected Doppler fetal heart sound, the band pass filter having a pass band from f_{L} to f_{H};

a time-frequency analysis module configured to apply time-frequency analysis to the Doppler fetal heart sound pre-processed in the step S1 to obtain a time-frequency graph;

a characteristic band and template selection module configured to select a characteristic band, from f_{CL} to f_{CH}, in the signal from the time-frequency graph, and select a time-frequency block containing features of S1 sound and S2 sound from the time-frequency graph, the time-frequency block having a time interval of 0.2 seconds<t₀<0.5 seconds;

a cross-correlation module configured to calculate a cross-correlation function between the characteristic band and a template and plot a correlation curve based on a result of the cross-correlation function;

a peak extraction module configured to calculate a peak of the cross correlation curve by means of threshold detection; and

an instantaneous heart rate graph plotting module configured to calculate an instantaneous heart rate value by calculating a differential of the peak, and plot an instantaneous heart rate graph based on the instantaneous heart rate value.

Further, the band pass filter in the signal pre-processing module has the pass band of 50-250 Hz, and f_{L} is 50 Hz and f_{H} is 250 Hz.

Further, the time-frequency analysis module is configured to perform the time-frequency analysis by utilizing a Short Time Fourier Transform (STFT) defined as:

s(w,t)=1/2π∫_{-∞}^{+∞}e^{-iw}x(τ)h(τ−t)dτ (1)

where h(t) is a window function, x(τ) is a signal and r is a signal argument, t is a time variable and w is a frequency argument, wherein, by moving an analysis window along a time axis, the resulting two-dimensional time-frequency graph is represented as s(w,t).

Compared with the conventional solutions, the solutions according to the present disclosure have the following advantageous effects. In the method for detecting an instantaneous heart rate according to the present disclosure, a one-dimensional non-stationary fetal heart sound signal is converted to a two-dimensional time-frequency plane capable of describing variations of the signal frequency over time based on time-frequency analysis. Then, a characteristic template is extracted on the two-dimensional time-frequency plane based on a priori information on S1 sound and S2 sound. A normalized cross correlation curve between the characteristic template and a characteristic band is calculated, so as to calculate the instantaneous heart rate. The detection method according to the present disclosure has a higher accuracy than the conventional solutions.

### BRIEF DESCRIPTION OF THE DRAWINGS

FIG. 1 is a flowchart illustrating a method for detecting an instantaneous heart rate according to the present disclosure;

FIG. 2 is a schematic diagram showing an ultrasound Doppler fetal heart sound signal collected clinically;

FIG. 3 is a schematic diagram showing a two-dimensional time-frequency plane after time-frequency conversion of a fetal heart sound signal using STFT;

FIG. 4 is a schematic diagram showing a normalized cross correlation curve; and

FIG. 5 is a schematic diagram showing an instantaneous heart rate of a fetal heart sound signal detected using the detection method according to the present disclosure.

### DETAILED DESCRIPTION OF THE EMBODIMENTS

In the following, the solutions according to the present disclosure will be further explained with reference to the figures and embodiments.

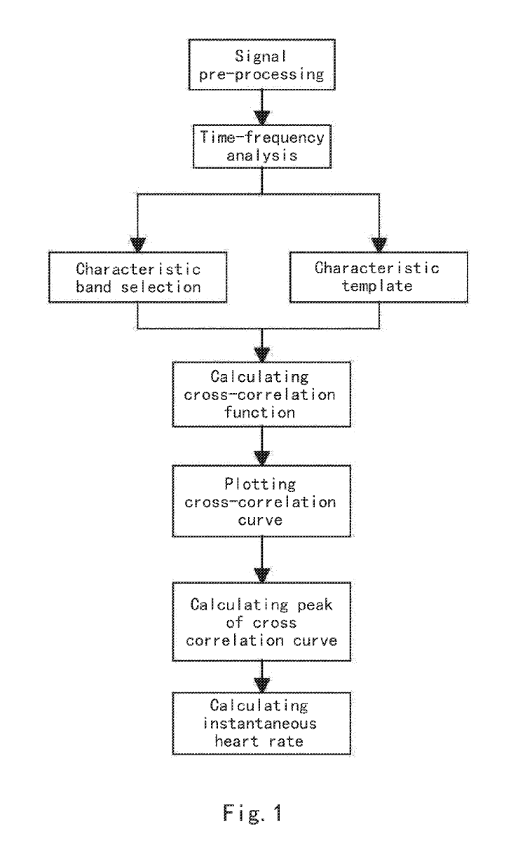

As shown in FIG. 1, a method for detecting an instantaneous fetal heart rate of a Doppler fetal heart sound based on time-frequency analysis according to the present disclosure includes the following steps.

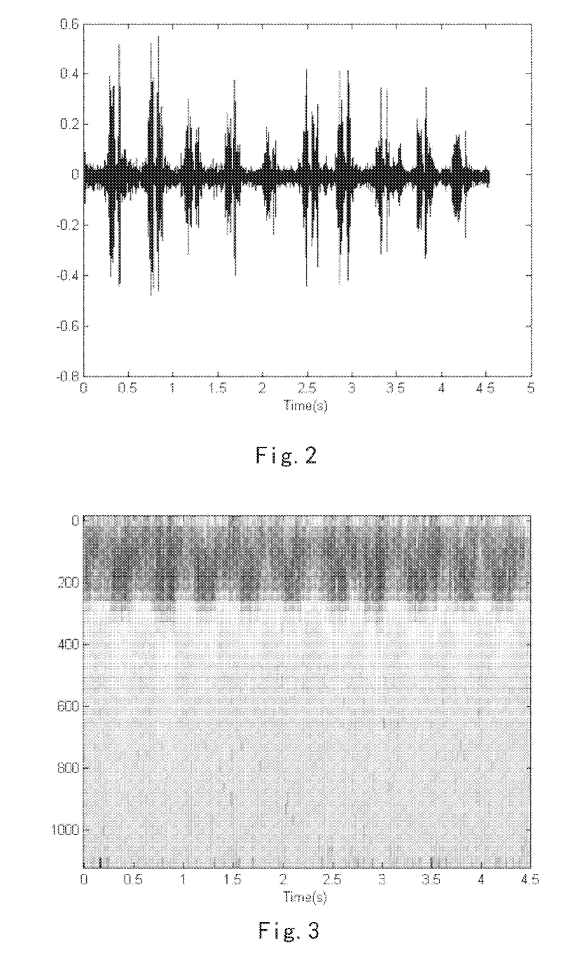

S1—Signal pre-processing: A band pass filter is applied to a collected Doppler fetal heart sound. The band pass filter has a pass band from f_{L} to f_{H}. The collected Doppler fetal heart sound signal is shown in FIG. 2. In this embodiment, the band pass filter has the pass band of 50-250 Hz, i.e., f_{L} is 50 Hz and f_{H} is 250 Hz.

S2—Time-frequency analysis: Time-frequency analysis is applied to the Doppler fetal heart sound pre-processed in the step S1. According to the present disclosure, the time-frequency analysis is performed by utilizing a Short Time Fourier Transform (STFT) to obtain a two-dimensional time-frequency plane graph as shown in FIG. 3. The STFT is a time-frequency analysis method defined as:

s(w,t)=1/2π∫_{-∞}^{+∞}e^{-iw}x(τ)h(τ−t)dτ (1)

where h(t) is a window function, x(τ) is a signal and τ is a signal argument, t is a time variable and w is a frequency argument. By moving an analysis window along a time axis, the resulting two-dimensional time-frequency graph is represented as s(w,t).

S3—Characteristic band selection: For the time-frequency graph shown in FIG. 3, a characteristic band of 200-400 Hz is selected from the signal, i.e., f_{CL} is 200 Hz and f_{CH} is 400 Hz.

Template selection: For the time-frequency graph shown in FIG. 3, a time-frequency block containing features of S1 sound and S2 sound is selected from the time-frequency graph. It is to be noted that the time interval to of the time-frequency block should satisfy: 0.2 seconds<t₀<0.5 seconds. In this embodiment, the time interval of the time-frequency block t₀=0.4 seconds.

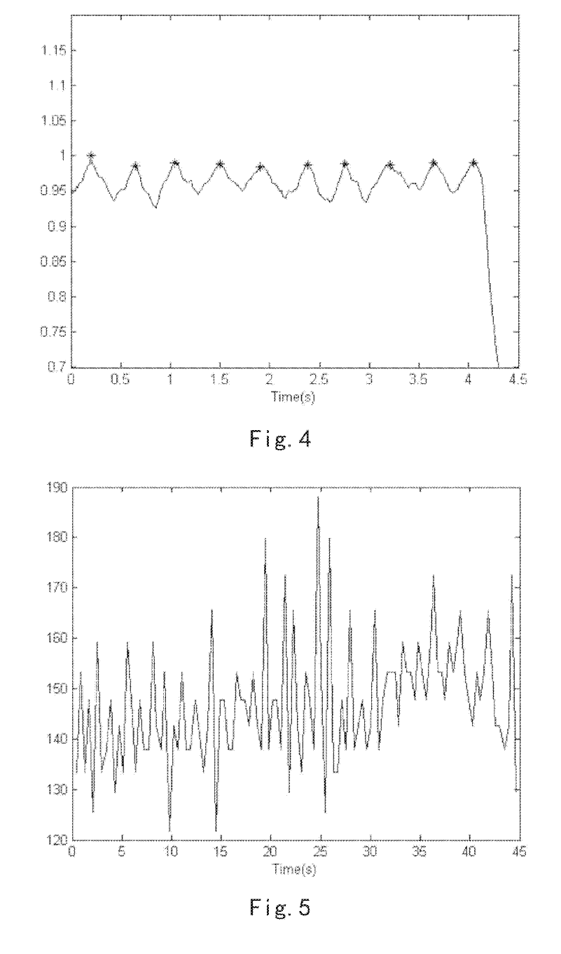

S4—Cross-correlation function calculation: A cross-correlation function between the characteristic band and the template as obtained in the step S3 is calculated. A cross correlation curve is plotted based on a result of the cross-correlation function. The plotted cross correlation curve is shown in FIG. 4. Here, a two-dimensional cross correlation function is as follows:

$$C ( i , j ) = {\sum }_{m = 0}^{Ma - 1} {\sum }_{n = 0}^{Na - 1} A ( m , n ) \cdot conj ( B ( m + i , n + j ) ) \qquad ( 2 )$$

where A is a Ma×Na matrix, B is a Mb×Nb matrix, conj(B) denotes a conjugate of B, 0≤i<Ma+Mb−1, 0≤j<Na+Nb−1, and C(i,j) denotes the cross correlation curve.

S5: A peak of the cross correlation curve is calculated by means of threshold detection. In this embodiment, the threshold is:

threshold=param×max{R(n)},

wherein param is value ranging from 0 to 1, and R(n) denotes the cross correlation curve. In an embodiment, param can be a value equal to or larger than 0.9.

S6: An instantaneous heart rate value is calculated by calculating a differential of the peak. Here, the instantaneous heart rate is calculated as:

$$\begin{matrix}Instantaneous Heart Rate = \frac{60}{Time Interval between Two Adjacent Peaks ( seconds )} ( beats \text{/} second ) & ( 3 ) \\ or & \\ Instantaneous Heart Rate = \frac{6000}{Time Interval between Two Adjacent Peaks ( ms )} ( beats \text{/} min ) & ( 4 )\end{matrix}$$

In this embodiment, the instantaneous heart rate is calculated using Equation (3). An instantaneous heart rate graph is plotted based on the instantaneous heart rate value, as shown in FIG. 5.

Obviously, the above embodiments are only examples for explaining the present disclosure clearly, rather than limiting the present disclosure. The embodiments are not exhaustive and various modifications or alternatives can be made to the above embodiments by those skilled in the art. All modifications, equivalents and improvements made without departing from the spirit and principle of the present disclosure are to be encompassed by the scope of the present disclosure, which is defined by the claims as enclosed.

What is claimed is:

1. A method for detecting an instantaneous fetal heart rate of a Doppler fetal heart sound based on time-frequency analysis, comprising steps of:
S1—signal pre-processing: applying a band pass filter to a collected Doppler fetal heart sound, the band pass filter having a pass band from f_{L} to f_{H};
S2—time-frequency analysis: applying time-frequency analysis to the Doppler fetal heart sound pre-processed in the step S1 to obtain a time-frequency graph;
S3—characteristic band and template selection: selecting a characteristic band, from f_{CL} to f_{CH}, in the signal from the time-frequency graph, and selecting a time-frequency block containing features of S1 sound and S2 sound from the time-frequency graph, the time-frequency block having a time interval of 0.2 seconds<t₀<0.5 seconds;
S4—cross-correlation function calculation: calculating a cross-correlation function between the characteristic band and a template and plotting a correlation curve based on a result of the cross-correlation function;
S5: calculating a peak of the cross correlation curve by means of threshold detection; and
S6: calculating an instantaneous heart rate value by calculating a differential of the peak, and plotting an instantaneous heart rate graph based on the instantaneous heart rate value.

2. The method of claim 1, wherein, in the step S1, f_{L} is 50 Hz and f_{H} is 250 Hz, and the band pass filter has the pass band of 50-250 Hz.

3. The method of claim 1, wherein, in the step S2, the time-frequency analysis is performed by utilizing a Short Time Fourier Transform (STFT) defined as:
s(w,t)=1/2π∫_{-∞}^{+∞}e^{-iw}x(τ)h(τ−t)dτ (1)
where h(t) is a window function, x(τ) is a signal and r is a signal argument, t is a time variable and w is a frequency argument, wherein, by moving an analysis window along a time axis, the resulting two-dimensional time-frequency graph is represented as s(w,t).

4. The method of claim 1, wherein, in the step S3, the characteristic band is 200-400 Hz, and f_{CL} is 200 Hz and f_{CH} is 400 Hz.

5. The method of claim 1, wherein, in the step S4, the correlation curve is plotted by utilizing a two-dimensional cross correlation function as:
$$C ( i , j ) = {\sum }_{m = 0}^{Ma - 1} {\sum }_{n = 0}^{Na - 1} A ( m , n ) \cdot conj ( B ( m + i , n + j ) ) \qquad ( 2 )$$
where A is a Ma×Na matrix, B is a Mb×Nb matrix, conj(B) denotes a conjugate of B, 0≤i<Ma+Mb−1, 0≤j<Na+Nb−1, and C(i,j) denotes the cross correlation curve.

6. The method of claim 1, wherein, in the step S5, the peak of the cross correlation curve is calculated by means of threshold detection, wherein the threshold is:
threshold=param×max{R(n)},
wherein param is 0.9 or a value close to 0.9, and R(n) denotes the cross correlation curve.

7. The method of claim 1, wherein, in the step S6, the instantaneous heart rate is calculated as:
$$\begin{matrix}Instantaneous Heart Rate = \frac{60}{Time Interval between Two Adjacent Peaks ( seconds )} ( beats \text{/} second ) & ( 3 ) \\ or & \\ Instantaneous Heart Rate = \frac{6000}{Time Interval between Two Adjacent Peaks ( ms )} ( beats \text{/} min ) & ( 4 )\end{matrix}$$

8. An apparatus for applying the method for detecting an instantaneous fetal heart rate of a Doppler fetal heart sound based on time-frequency analysis according to claim 1, comprising:
a signal pre-processing module configured to apply a band pass filter to a collected Doppler fetal heart sound, the band pass filter having a pass band from f_{L} to f_{H};
a time-frequency analysis module configured to apply time-frequency analysis to the Doppler fetal heart sound pre-processed in the step S1 to obtain a time-frequency graph;
a characteristic band and template selection module configured to select a characteristic band, from f_{CL} to f_{CH}, in the signal from the time-frequency graph, and select a time-frequency block containing features of S1 sound and S2 sound from the time-frequency graph, the time-frequency block having a time interval of 0.2 seconds<t₀<0.5 seconds;
a cross-correlation module configured to calculate a cross-correlation function between the characteristic band and a template and plot a correlation curve based on a result of the cross-correlation function;
a peak extraction module configured to calculate a peak of the cross correlation curve by means of threshold detection; and
an instantaneous heart rate graph plotting module configured to calculate an instantaneous heart rate value by calculating a differential of the peak, and plot an instantaneous heart rate graph based on the instantaneous heart rate value.

9. The apparatus of claim 8, wherein the band pass filter in the signal pre-processing module has the pass band of 50-250 Hz, and f_{L} is 50 Hz and f_{H} is 250 Hz.

10. The apparatus of claim 8, wherein the time-frequency analysis module is configured to perform the time-frequency analysis by utilizing a Short Time Fourier Transform (STFT) defined as:
s(w,t)=1/2π∫_{-∞}^{+∞}e^{-iw}x(τ)h(τ−t)dτ (1)
where h(t) is a window function, x(τ) is a signal and r is a signal argument, t is a time variable and w is a frequency argument, wherein, by moving an analysis window along a time axis, the resulting two-dimensional time-frequency graph is represented as s(w,t).

11. The apparatus of claim 9, wherein the time-frequency analysis module is configured to perform the time-frequency analysis by utilizing a Short Time Fourier Transform (STFT) defined as:
s(w,t)=1/2π∫_{-∞}^{+∞}e^{-iw}x(τ)h(τ−t)dτ (1)
where h(t) is a window function, x(τ) is a signal and τ is a signal argument, t is a time variable and w is a frequency argument, wherein, by moving an analysis window along a time axis, the resulting two-dimensional time-frequency graph is represented as s(w,t).
